(19) United States
(12) Patent Application Publication
(10) Pub. No.: US 2005/0037081 A1
(43) Pub. Date: Feb. 17, 2005

(54) Novel formulation

(76) Inventors: Gillian Eccleston, Glasgow (GB); Ronald Paterson, Leicestershire (GB)

Correspondence Address: Janis K Fraser;Fish & Richardson, 225 Franklin Street, Boston, MA, 02110-2804, US

(21) Appl. No.: 10/493,711

(22) PCT Filed: Oct. 29, 2002
(86) PCT No.: PCT/SE02/01956

(30) Foreign Application Priority Data
Oct. 30, 2001 (SE) 0103613-6

(51) Int. Cl. A61K 9/14

(52) U.S. Cl. 424/488; 514/54

(57) ABSTRACT

The present invention relates to a multiple unit, floating dosage form for oral administration, comprising an alginic acid salt, pectin and a pharmaceutically active ingredient. The pharmaceutically active ingredient can for example be an H2 antagonist, an antibiotic, an antibacterial or an antifungal agent. The pectin is preferably a high ester pectin, such as high methoxy pectin and the alginic acid can for example be selected from sodium or potassium alginate. The dosage form can be used in the topical treatment of deseases of the gastrointestinal tract.

The present invention relates to a novel, multiple unit, sustained release dosage form for oral administration and the preparation and use thereof.

### BACKGROUND OF THE INVENTION

Whilst being a convenient method for the delivery of therapeutic agents, oral administration can result in poor bioavailability and drug wastage. Rapid gastric emptying can result in low oral bioavailability especially when the administered therapeutic agent is only readily soluble in the low pH of the stomach and/or is absorbed only in a narrow region in the proximal part of the gastrointestinal tract. Further, oral administration suffers from the added problem that absorption time varies from patient to patient as gastric pH and drug dissolution time vary thus leading to unpredictable times to achieve peak plasma levels.

Optimisation of oral delivery in terms of imnproving oral bioavailability and consistency of absorption and reducing drug wastage may be achieved by prolonging the time spent by the therapeutic agent in the gastric environment.

The art teaches of several methods for prolonging the gastric retention of dosage forms. One such method involves the use of floating dosage forms which remain buoyant on the gastric fluids by virtue of their low density. Like other gastric retention dosage forms they provide for better dissolution of the therapeutic agent in the stomach, better absorption at the intestinal site of absorption and allow topical treatment of the gastrointestinal tract as well as systemic treatment of deseases.

Floating dosage forms are commercially available as single unit and multiple unit floating systems. Madopar (Roche Products, Welwyn Garden City, UK) is an example of a single unit system and comprises levodopa and benserazide as a hydrodynamicallybalanced system formulation which allows plasma levels of levodopa to remain consistent. However single unit floating systems are subject to “all or nothing” gastric emptying, a problem which can fortunately be avoided through the use of multiple unit dosage forms. In addition, multiple units provide for a more uniform distribution of the therapeutic agent through the gastrointestinal tract.

Whitehead et al. (Eur. J. Pharm. Sci. 4 (Supp): S182 and J. C6ntrolled. Release. 55 3-12) have designed multiple unit floating dosage forms based on calcium algmiate beads. These beads, when freezedried during the manufacturing process, have been found to float on gastric fluids by virtue of the porous system created on frieezedrying They can thus be retained in the stomach for more than 5.5 hours. Manuacture of the beads involves dropping sodium alginate into a solution or suspension of calcium chloride and a therapeutic agent. This causes precipitation of calcium alginate beads -contasning the therapeutic agent, which can then be freeze dried to form a floating dosage form.

However the range of therapeutic agents that can incorporated into these beads is limited by the fact that the calcium ions of this formulation can interact with ctain pharmaceutical ingredients to reduce their absorption by the body. It would therefore be beneficial to develop a dosage form with a composition which did not comprise calcium ions such that pharmaceutical ingredients not compatible with calcium ions could be incorporated.

In accordance with the present invention there is therefore provided a multiple unit floating dosage form comprising an alginic acid salt, pectin and a pharmaceutically active ingredient where the pharmaceutically active ingredient can be compatible or incompatible with calcium ions.

### SUMMARY OF INVENTION

A multiple unit floating dosage form comprising an alginic acid salt, pectin and a pharmaceutically active ingredient for oral administration is provided.

A process for the preparation of a multiple unit floating dosage form comprising an alginic acid salt, pectin and a pharmaceutically active ingredient is disclosed which process includes the dropwise addition of a biopolymer solution or suspension of an alginic acid salt and pectin also containing a soluble or insoluble therapeutic agent into acid; separation of the resulting hydrogel beads from the medium; snap freezing in liquid nitrogen; and freeze-drying.

A multiple unit floating dosage form comprising an alginic acid salt, pectin and a pharmaceutically active ingredient for oral administration is provided.

### DETAILED DESCRIPTION OF THE INVENTION

According to the present invention there is provided a multiple unit floating dosage form comprising an alginic acid salt, pectin and a pharmaceutically active ingredient suitable for oral administration.

Whitehead et al. disclose multiple unit floating dosage forms based on calcium alginate beads that can be retained in the stomach for more than 5.5 hours. However the use of these beads is limited by the fact that many pharmaceutically active ingredients are not compatible with calcium ions.

According to D. Thom et al. (Prog. Fd. Nutr. Sci, 6, 97-108) biopolymer solutions of alginates and pectins will form firm resilient gels, in the absence of calcium or high concentrations of sugars, under conditions of low pH.

We have surprising found that multiple unit floating dosage forms can be manufactured from biopolymer solutions of alginates and pectins into which can also be incorporated a pharmaceutically active ingredient that will be compatible with a wider range of pharmaceutically active ingredients than those previously disclosed in the art. In pati,cular the present invention shall be usefuil for the oral administration of pharmaceutically active ingredients that are not compatible with calcium or calcium ions. In addition, the dosage formis of the present invention have prolonged floatation and a consistent release profile for pharmaceutically active ingredients over a wide range of pH (pH 1 to 5). This pH range covers the pH range expected in both the fed and fasted states of the han stomach

Further to this, drug release times for the dosage forms of the present invention can be varied by varying the degree of association between the alginic acid salt and pectin within the hydrogel system. This can be achieved by varying the ratio of alginic acid salt to pectin in the formulation or by varying the pH at which the biopolymers are acidified to fbrm hydrogels.

The invention therefore provides for floating multiple unit dosage forms comprising an alginc acid salt, pectin and a pharmaceutically active ingredient.

Preferably, pectin is high ester pectin containing an ester content of greater than 50% along the biopolymer chains. More preferably pectin is high methoxy pectin.

Preferably, the alginic acid salt used is selected from potassium or sodium alginste. More. preferably, sodium alginate is used.

The pharmaceutically active ingredient can be selected from a wide range of dmgs, and may include H₂ antagonists, antibiotic, antibacterial and antifugal agents.

Examples of pharmaceutically active ingredients which have a reduced absorption in the body on interaction with calcium ions are biphosphonates such as aldendronic acid and disodium etidronate and antibacterials such as ciprofloxacin and tetracyclines. Tetrcyclines include doxycycline and oxytetracycline. Consequently the present invention will be usefull for the oral administration of these drugs.

Further the present invention will also be useful for increasing the bioavailability of drugs with narrow absorption windows in the upper small intestine such as fusemide, cyclosporin, allopurinol and ciprofloxacin and drugs that are poorly soluble in or not suited tothe higher pH of the small intestine. Such drugs also include diazepam, chlordiazepoxide, cinnanzine and captopril.

In a preferred embodiment, the dosage form is manufactured from a biopolymer solution or suspension in which sodium alginate is present at a concentration of 1% w/v and high to methoxy pectin at 1% w/v. The solution or suspension also contains the pharmaceutically active ingredient at in an appropriate amount.

The invention further provides fbr a process for the manufacture of ihe multiple unit dosage form.

To an aqueous solution of alginic acid salt and pectin, at the appropriate concentrations, is added the required amount of a phnaceutically active ingredient. The pharnaceutically active ingredient may be soluble or insoluble. The resultizg solution or suspension is then added dropwise to acid to yield hydrogel beads that are separated from the medium, frozen and freezedried. The material can be frozen using commonly known techniques for freeze drying including conventional freeze drying, preferably the matrisl is frozen by snap-freezing in liquid nitrogen. The resulting beads are loaded with the pharmaceutically active ingredient. Suitably the bead size is firom about 0.1 to about 10 mm, preferably the bead size is about 2.5 mm.

The beads can range in size from about 1 to about 8 mm in diameter. Beads with a diameter of about 3 to about 4 mm are preferred and most preferred are beads with a diameter of about 3.15 mm

Preferably, the alginic acid salt used in the process is selected from potassium of sodium alginate. More preferably, sodium alginate is used.

Preferably, pectin is selected from high ester pectins containing aii ester content of greater than 50% along the biopolymer chains. More preferably high methoxy pectin is used.

The hydrogel beads can be snap frozen by means known in the art, for example by submerging them in liquid nitrogen.

The conditions for freeze-drying may, for example, be a temperature of −40° C. for 24 hours.

In a preferred embodiment, the biopolymer solution or suspension to be acidified and form a hydrogel substance is such that sodium alginate is present at a concentration of 1% w/v and the high methoxy pecn at 1% w/v. The solution or suspension also contains the is pharnaceutically active ingredient at in an appropriate amount.

The dosage form of the present invention is also usefuil for the topical treatment of the gastrointestinal tract. In particular the dosage form may be useful for the delivery of pharmaceutically active ingredients to the upper gastrointestinal mucosa to treat, for example, helicobacter pylori. Further, the present invention also allows the systemic treatment of disease. There is therefore provided the.dosage form of the present invention for use in therapy and a method of treating a patient in need of therapy, comprising administering to said patient a dosage form of the present invention containing an appropriate therapeutic agent.

### EXAMPLS

### Example 1

Sodium alginate (1% w/v) and high ester pectin containg a methyl ester content of greater than 50% were dissolved in distilled water. The insoluble compound griseofulvin was suspended in the biopolymer solution. The suspension was then added drop-wise from a syringe (1 inch from the surface) with a 21-G needle attachment, to 500 ml of HCl (vortex mixed) across a range of concentrations from 0.01M to 0.1M. Drug loaded hydrogel beads formed. The beads were separated from the medium, snap frozen in liquid nitrogen and freeze-dried at −40° C. for 24 hours. The loading efficiency of griseofulvin was 97%.

### Example 2

Sodium alginate (1% w/v) and high ester pectin containg a methyl ester content of greater than 50% were dissolved in distilled water. The insoluble compound paracetamol was dissolved in the biopolymer solution. The suspension was then added drop-wise from a syringe (1 inch from the surface) with a 21-G needle attachment, to 500 ml of HCl (vortex mixed) across a range of concentrations from 0.01M to 0.1M. Drug loaded hydrogel beads formed. The beads were separated from the medium, snap frozen in liquid nitrogen and freeze-dried at −40° C. for 24 hours. The loading efficiency of paracetamol was 40%.

### Example 3

The freeze-dried beads of example 1 were treated for prolonged floatation over a 12 hour period in vitro on distilled water and on simulated gastric fluid with pH ranging for pH 1 to pH 5. Floatation was independent of pH.

The drug release profile was also recorded and found to be consistent over this in vitro pH range.

1. A multiple unit floating dosage form comprising an alginic acid salt, a pectin and a pharmaceutically active ingredient.

2. A dosage form according to claim 1 wherein the pectin is a high ester pectin containing an ester content of greater than 50% along the biopolymer chains.

3. A dosage form according to claim 2 wherein the pectin is high methoxy pectin.

4. A dosage form according to claim 3 wherein the dosage form is manufactured from a solution or suspension comprising high methoxy pectin at a concentration ranging from 0.5 to 5% w/v, preferably 0.5 to 1.5% w/v.

5. A dosage form according to claim 4 wherein the high methoxy pectin is present at a concentration of 1% w/v.

6. A dosage form according to claim 1 wherein the alginic acid salt is selected from sodium alginate and potassium alginate.

7. A dosage form according to claim 6 wherein the alginic acid salt is sodium alginate.

8. A dosage form according to claim 7 wherein the dosage form is manufactured from a solution or suspension comprising sodium alginate at a concentration ranging from 0.5 to 5% w/v, preferably 0.5 to 1.5% w/v.

9. A dosage form according to claim 8 wherein sodium alginate is present at a concentration of 1% w/v.

10. A dosage form according to claim 1 wherein the pharmaceutically active ingredient is selected from H₂ antagonists, antibiotic, antibacterial and antifungal agents.

11. A dosage form according to claim 1 wherein the pharmaceutically active ingredient is selected from:
active ingredients which have a reduced absorption in the body on interaction with calcium ions;
active ingredients with narrow absorption windows in the upper small intestine; and
active ingredients that are poorly soluble in or not suited to the higher pH of the small intestine.

12. A dosage form according to claim 1 wherein the pharmaceutically active ingredient is selected from biphosphonates such as aldendronic acid and disodium etidronate, tetracyclines such as doxycycline and oxytetracycline, frusemide, cyclosporin, allopurinol, ciprofloxacin, diazepam, chlordiazepoxide, cinnarizine and captopril.

13. A dosage form according to claim 1 wherein the multiple unit comprises beads.

14. A dosage form according to claim 13 wherein the beads have a diameter ranging from about 1 mm to about 8 mm.

15. A dosage form according to claim 14 wherein the beads have a diameter of about 3.15 mm.

16. A process for the manufacture of a dosage form according to claim 1 comprising:
adding the required amount of a pharmaceutically active ingredient to an aqueous solution of alginic acid salt and pectin at the appropriate concentrations;
dropwise addition of the resulting solution or suspension to acid;
separation of the resulting hydrogel beads from the medium; and
snap-freezing and freeze drying of the hydrogel beads.

17. (Cancelled)

18. (Cancelled)

19. A method of treating a patient in need of therapy, comprising administering to said patient a dosage form as claimed in claim 1.

20. The method of claim 19, wherein the patient is in need of treatment of a disease of the gastrointestinal tract.
